(19) United States
(12) Patent Application Publication
(10) Pub. No.: US 2023/0046773 A1
(43) Pub. Date: Feb. 16, 2023

(54) System and method of on-demand mobile Intravenous (IV) Hydration APP for hangover cures, vitamin infusions, and other acute illness

(71) Applicant: Sachmarie Roberta Crowley, Saratoga Springs, NY (US)

(72) Inventor: Sachmarie Roberta Crowley, Saratoga Springs, NY (US)

(21) Appl. No.: 17/402,514

(22) Filed: Aug. 14, 2021

(51) Int. Cl. G16H 20/17; G16H 80/00; G16H 40/67; G16H 10/60

(52) U.S. Cl. CPC G16H 20/17; G16H 80/00; G16H 40/67; G16H 10/60; G06Q 30/04

(57) ABSTRACT

This disclosure relates generally to a system and method for the optimized management of health care services provision with fulfilment of prescription orders for medications and health-related products in a health care environment. A computer-controlled system coordinates and controls, on demand IV hydration treatments or acute illness management fulfilment in a most efficient path thereby minimizing a cost and time function associated with fulfilment of the order. A smartphone, a laptop computer, a portable communicating device, a cloud computing device, or a data storage device is capable of communicating with the computer. The proposed healthcare management system-connects care providers, patients and delivery teams for quick, clear, and efficient communication and feedback/follow up features. It aims in reducing errors of, unawareness about and time & effort for seeking medical/health care. In order words, it makes a care-need assessment, order placement, fulfilment and status tracking a breeze.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever

### BACKGROUND

### Field of the Invention

Embodiments relate to an online, data processing systems and more particularly to data processing systems used to manage delivery of healthcare services. The present invention further relates to methods and an apparatus for on-demand Intravenous Fluids and vitamins infusions and injections at the request of the customer.

### Description of the Related Art

Typically, when a person suffers an illness or injury, that person must have transportation to a medical facility for diagnosis and treatment of the illness or injury. Managed healthcare is an important service provided to various non-medical facilities or institutions, such as, for example, correctional facilities, remote military or scientific bases, and large ships e.g., factory and cruise ships, etc., that have a patient clinic and/or a patient infirmary. Most of such facilities do not, however, have the internal resources to fully provide for all the branches of patient care. Further, smaller communities may be unable to afford a properly staffed medical facility or hospital, may not have enough of a population to support various medical specialties typically serviced by specialist physicians, or may have both adequate finding and population but nevertheless may have an inadequately staffed medical facilities due to physician recruiting difficulties.

In less modern times, the delivery of medical services did not require the level of training and education that is required today. For example, historically, most severing wound infections were treated through amputation rather than application of a regiment of antibiotics and/or localized surgery. Due to the advances in modern medicine, the number of illnesses and injuries that can now be treated have significantly increased while the number of properly trained in educated physicians, especially specialist physicians, have not kept pace. As a result, the difficulty of providing timely and cost-efficient patient access to such medical professionals has become increasingly complicated, especially in the various non-medical facilities, institutions, and communities, described above, that are unable to support their own fully staffed hospital staffed with a sufficient number of physicians to provide adequate coverage for each medical specialty.

Even through the ease of reaching to medical staff and multiple modes of access to medical information is available, generally there is still an insufficient pool of medical services available to every individual.

Further, even when there are adequate medical resources, individual's transportation problem and in current epidemic situation the access to medical services is always a matter of concern. Furthermore, a significant amount of patient's time is wasted in transporting between their “home” location and the location of the patient. Patients are often either need to be transported great distances in order to obtain such services which aren't suitable for patient's health.

By looking at prior art there are multiple systems that have been proposed in prior art regarding health care systems. For instance, a CA patent 2,875,349A1 on Systems and Methods for Processing House Calls discloses systems and methods for scheduling and processing house calls between a plurality of patients and a plurality of practitioners, The systems and methods use a client-server architecture, removing the need for a call center to perform patient intake and house call scheduling, and improving workflows by allowing practitioners to view and select patient requests for house calls. Other embodiments relate to systems and methods for generating claim codes and diagnostic codes from a set of practitioner notes, and transmitting them to a third party for processing.

Another JP patent 2,014,229245A on House Call State Confirmation Support System and Method allows a house call user and the like to decide at-home time, arrangement time of a courtesy car, and other schedules according to visit time of a medical doctor by understanding a house call state of the medical doctor, for improving convenience for the house call user and other medical care participants in a house call medical care support system. SOLUTION: A house call state confirmation support device can be connected to a user terminal 30 and a medical doctor terminal 40. The support device comprises: a registration part 15; a presentation part 14; and a database 16. The database 16 stores an order 81 of house call visit destinations where the medical doctor having the medical doctor terminal 40 visits for house call, positions 82 of the house call visit destinations, distance 83 between the house call visit destinations whose orders are adjacent, and medical care completion states 84 of respective house call visit destinations.

Another US patent 2,013,0183923A1 on Field nurse notification is issued to Honeywell International Inc. The patent discusses a system and method (10) are disclosed for providing home health care to a plurality of home health care patients (12) by utilizing the services of field nurses (14). The system (10) includes at least one centralized programmable processor (16) configured to respond to a field alert request (18) associated with one of the patients (12) by accessing an electronic database (20) for a patient dataset (22) specific to the patient (12) and automatically initiating a field alert in the form of a wireless communications signal (24) transmitting a portion or all of the patient dataset (22) to a wireless communications device 26 assigned to a field nurse (14).

Another US patent 2,012,0290311A1 Method and Apparatus for Physician Location Tracking is issued to PRANA TECHNOLOGY Inc. The patent allows a medical personal location tracking system provides efficient physician location reporting based on physician client device location. The medical communication system permits physicians to define location tracking control settings to control when and where the client device sends location data to a server and database. Subject to the location tracking control settings, the location tracking system provides location updates for the physicians so that requestors that wish to contact the physicians or know the location of one or more physicians can access the server to obtain location data. To obtain medical assistance to a medical emergency, an emergency alert may be generated and sent to physicians within a defined geographic area. Upon receipt by a physician, a physician may accept the emergency alert and travel to the medical emergency and be provided updates regarding the medical emergency. The person generating the emergency alert may track the location of accepting physicians.

A KR20190060902A Digital house call service platform relates to a digital house call service platform which connects medical customers, such as a patient, with medical suppliers to enable the medical customer to receive a visit medical service from the medical supplier at a desired place. According to the present invention, the digital house call service platform comprises: a house call bag carried by house call medical staff; a medical laptop computer embedded in the house call bag and transmitting medical information of a customer to a hospital; and a plurality of diagnostic devices embedded in the house call bag and connected to the medical laptop computer.

A U.S. Pat. No. 7,249,036B2 on Method for clinician house calls utilizing portable computing and communications equipment is issued to Bayne Gresham. The patenty discusses a mobile clinicians conduct in-home patient visits utilizing on-site diagnostic and treatment equipment, where service is enhanced by the use of portable computing and communications equipment. Initially, a mobile care entity provides a network of predesignated mobile clinicians, each having the use of a preprogrammed portable computer. Each computer is coupled to a wireless communications device, and includes local storage of patient data. Whenever the mobile care entity receives requests for medical service at a patient's premises, the entity selects a mobile clinician and dispatches him/her to the patient's premises. The clinician visits the patient's premises accompanied by an assortment of electronic diagnostic and treatment devices. To examine the patient, the doctor utilizes various diagnostic devices to prepare machine-readable reports of related aspects of the patient's condition.

There are multiple inventions that have been proposed in prior art regarding effective healthcare management and delivery system. However, the utility of these systems has not been seen in advance form and for specialized needs. Currently most existing and traditional health care services to patients is a complicated process that requires cohesion and coordination amongst care-providers. Currently, this process is segregated into care providers' functional and locational silos resulting in inter-functional and inter-locational lack effective and efficient coordination and access. This is particularly true in the context of health care of patients with regular medical demands, where the obvious time constraints often preclude adequate, and sometimes extremely critical, intervention and/or fulfilment by other care providers. Sometimes, it results in a care decision which is not cost-effective, clinically-suited or safe. Sometimes, resulting order for medical care cannot be fulfilled owing to time or stock constraints.

The current invention proposes a system where a new approach, utilizing the best of breed in existing technologies, to revolutionize the digital arena, implementing the bridge that fills the gap between hospitals and patients is implemented. This mobile APP is for anyone requesting intravenous hydration and vitamin infusions and injections. The targeted customers are ones that are suffering from hangovers, migraines, nausea, vomiting, diarrhea or any other acute ailments.

The proposed platform seeks to significantly simplify the whole process and efficiently manages the circumstances through its swift ability to connect care providers. The invention relates to the development of an automated healthcare system that addresses the needs and desires of both healthcare providers (e.g., clinicians, pharmacists, nurses and health professionals) and patients. The system encompasses computer communications network-based systems, software, various input and output stations in order to treat patients by the provision of an updated health care system which is align with HIPAA compliant. The system also aims to presents multiple additional features which can provide advance level of assistance to its users.

As per applicants' knowledge, none of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is therefore the purpose of the invention to alleviate at least to some extent one or more of the aforementioned problems of the prior art and/or to provide the relevant public with a suitable alternative thereto having relative advantages.

The primary object of the invention is related to the provision of an automated healthcare system that involves both healthcare providers (e.g., clinicians, pharmacists, nurses and allied health professionals) and patients. The system encompasses computer communications network-based systems, software, various input and output stations in order to provide patients the provision of an updated health care system at their doorstep.

It is also the objective of the invention to provide a simplified process of health care information system including an information database and a health care doorstep services module. The information database is adapted to store a record for a patient including patient context data. The services module is adapted to receive required services order from the patient.

It is also the objective of the invention to provide a system which has provision of choosing delivery option be configured for requesting party. This can be set for specific time or date.

It is moreover the objective of the invention to provide a platform, where user can request an on demand mobile Intravenous (IV) Hydration APP for hangover cures, vitamin infusions, and other acute illness.

It is also the objective of the invention to provide a system where when creating a new account, the new member can send the invitation to new user. Upon signup through that invitation, an email or text will be sent to nurses and health care staff with the link of portal and/or app with login info. The patient will add or update their medical history and requirements for relevant services.

It is further the objective of the invention to provide a dual and reliable system which is easy to install and is easily compatible for all types of smart devices.

It is moreover the objective of the invention to provide a system which is inline with latest technologies and utilizes the latest health care information system including but not limited to real time environment, a highly compatible and artificial intelligence-based system is introduced.

It is further the objective of the invention to provide an interface which is easy to use, and where new users are provided with tutorials and necessary guidance to use the system.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The current invention in its preferred embodiment discloses a new approach of requesting healthcare services especially on demand mobile Intravenous (IV) Hydration APP for hangover cures, vitamin infusions, and other acute illness.

In embodiments, methods and systems for automatically maintaining an enhanced health care management system for a patient in accordance with various embodiments of the present invention may be provided. In embodiments, methods and systems may include an automatic data collection facility which refers to a server-based system compromising of a database compromising a real-time data integration facility. In embodiments, the assistance requirement services are initiated by the patient or caregiver of patient. The requirement can be of medical services including but not limited to provision of hangover cures, vitamin infusions, and other acute illness.

The health care management system has some features, which are further elaborated and defined for reference. In this embodiment, the term ‘Health care’ in this application refers to any and all services, whether explicitly mentioned or not, provided by a health care provider. For a way of example, not limitation, it includes prescription filling, compounding, counselling a patient, performing screening and diagnostic test and prescription delivery to the patient or to a facility where the patient is under care.

In the present system of healthcare management and administration method at patient's doorstep may facilitate ordering, dispensing, provision of services, reporting and billing.

The system as per its further embodiments allows configuring Delivery options for instance including but not limited to time or date. It also provides facility specific or other related indicators selection for the delivery of medicines to the required location.

The system as per its additional embodiments facilitates the process and improves on signup process. When creating a new account, it would be great to send the invitation to new user. Upon signup through that invitation, an email or text will be sent to patients and nurses with the link of portal and/or app with login information. Once a patient signs up, he/she will add medical history. Based on medical history and requirements mentioned by patient health care staff will be linked and services will be provided.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A health/medical care management system to provide care services to the patients where, the system comprising: a plurality of service application instances, devices under use by a plurality of healthcare provider, receive electronic orders, the server is configured to perform at least one of:
A method and system for communication between health care providers and patients for quick clear efficient and error-free real-time communication with feedback and follow up capabilities in the provision, assessment, tracking, and medical care services in a hierarchical manner;
A method of retrieving patient's medical details and determination of drug interaction information associated with the electronic orders;
A medication validation and billing facility in electronic communication with the medication management facility, the medication validation and billing facility configured to receive the electronic orders, provide information generating an order for pharmaceutical-care, transmitting such order to a single or plurality of health care staff and provision of required services to the patient;
A method of assigning delivery options based on multiple indicators including date and time;
A method of compiling, generating, exporting, printing, electronically transmitting treatment history reports with all details in accordance with regulatory requirements; and,
A method of providing tutorials and information regarding efficient use of the system.
